(19) United States
(12) Patent Application Publication
(10) Pub. No.: US 2009/0258824 A1
(43) Pub. Date: Oct. 15, 2009

(54) Amyloid beta receptor and uses thereof

(76) Inventors: Karen H. Ashe, North Oaks, MN (US); Sylvain E. Lesne, Minneapolis, MN (US); Eric A. Newman, Minneapolis, MN (US)

Correspondence Address: MUETING, RAASCH & GEBHARDT, P.A., P.O. BOX 581336, MINNEAPOLIS, MN, 55458-1336, US

(21) Appl. No.: 11/989,264

(22) PCT Filed: Jul. 27, 2006
(86) PCT No.: PCT/US2006/029452

§ 371 Date: Sep. 15, 2008

Related U.S. Application Data
(60) Provisional application No. 60/703,653, filed on Jul. 29, 2005.

(51) Int. Cl. A61K 38/16; G01N 33/53; A61P 25/28

(52) U.S. Cl. 514/12; 435/7.1

(57) ABSTRACT

The present invention relates to the identification that soluble assemblies of amyloid β function as a NMDA receptor antagonist. The present invention also provides methods and compositions for the detection and treatment of neurodegenerative and cognitive disorders and screening methods to identify agents that modulate the antagonistic effect of soluble assemblies of amyloid β on NMDA receptor function.

This application claims the benefit of U.S. Provisional Application Ser. No. 60/703,653, filed Jul. 29, 2005, which is incorporated by reference herein.

### GOVERNMENT FUNDING

The present invention was made with government support under Grant No. NS33249, awarded by the National Institutes of Health. The Government may have certain rights in this invention.

### BACKGROUND

The amyloid-β protein (AP) is implicated in the pathogenesis of Alzheimer's disease (AD). The AP peptides are the major amyloid protein deposited in AD brains and both natural and synthetic forms have devastating effects on the viability and function of neurons. See, for example, Yankner et al., Science 250, 279-82 (1990); Pike et al., Brain Res 563, 311-4 (1991); Pike et al., J Neurosci 13, 1676-87 (1993); Lambert et al., Proc Natl Acad Sci U S A 95, 6448-53 (1998); Walsh et al., Nature 416, 535-9 (2002); and Kayed et al., Science 300, 486-9 (2003). However, the mechanism by which the accumulation of Aβ proteins alters physiologic functioning in the brain and disrupts memory and cognitive function is unclear.

### SUMMARY OF THE INVENTION

The present invention includes a method of detecting a neurodegenerative disease and/or cognitive disorder in a subject, the method including obtaining a sample from the subject; immunoprecipitating the sample with an antibody to a NMDA receptor; wherein the coprecipitation of amyloid β along with the NMDA receptor indicates the subject has a neurodegenerative disease and/or cognitive disorder. In some embodiments, the amyloid β is a soluble assembly of amyloid β protein. In some embodiments, the antibody to an NMDA receptor is an antibody that binds to a NMDA receptor subunit selected from NR1, NR2A, and/or NR2B.

The present invention also includes a method of detecting a presymptomatic neurodegenerative disease and/or cognitive disorder in a subject, the method including obtaining a sample from the subject; immunoprecipitating the sample with an antibody to a NMDA receptor; wherein the coprecipitation of amyloid β along with the NMDA receptor indicates the subject has a presymptomatic neurodegenerative disease and/or cognitive disorder disease. In some embodiments, the amyloid β is a soluble assembly of amyloid β protein. In some embodiments, the antibody to an NMDA receptor is an antibody that binds to a NMDA receptor subunit selected from NR1, NR2A, and/or NR2B.

The present invention includes a method of inhibiting NMDA receptor function, the method including contacting a NMDA receptor with a soluble assembly of amyloid β protein.

The present invention includes a method of screening for an agent that alters the antagonistic effect of a soluble assembly of amyloid β protein on NMDA receptor function, the method including: contacting a NMDA receptor with the agent and a soluble assembly of amyloid β protein; determining NMDA receptor function for the NMDA receptor contacted with the agent and the soluble assembly of amyloid P protein; comparing NMDA receptor function for the NMDA receptor contacted with the agent and the soluble assembly of amyloid β protein to NMDA receptor function for a NMDA receptor contacted with the soluble assembly of amyloid β protein and not contacted with the agent; wherein a difference in the level of NMDA receptor function in the NMDA receptor contacted with the agent and the soluble assembly of amyloid β protein compared to NMDA receptor function in the NMDA receptor contacted with the soluble assembly of amyloid β protein and not contacted with the agent indicates the agent alters the antagonistic effect of the soluble assembly of amyloid β protein on NMDA receptor function. In some embodiments, the agent inhibits the antagonistic effect of the soluble assembly of amyloid β protein on NMDA receptor function. In some embodiments, NMDA receptor function is determined by whole cell patch clamp recording. In some embodiments, NMDA receptor function is determined by Ca⁺² fluorescence imaging. The present invention also includes agents identified by such methods.

The present invention includes a method of screening for an agent for the treatment of neurodegenerative disease and/or cognitive disorder, the method including: contacting a NMDA receptor with the agent and a soluble assembly of amyloid β protein; determining NMDA receptor function for the NMDA receptor contacted with the agent and the soluble assembly of amyloid β protein; comparing NMDA receptor function for the NMDA receptor contacted with the agent and the soluble assembly of amyloid β protein to NMDA receptor function for an NMDA receptor contacted with the soluble assembly of amyloid β protein and not contacted with the agent; wherein an altered level of NMDA receptor function in the NMDA receptor contacted with the agent and the soluble assembly of amyloid β protein compared to the NMDA receptor function of the NMDA receptor contacted with the soluble assembly of amyloid β protein and not contacted with the agent indicates the agent as an agent for the treatment of neurodegenerative disease and/or cognitive disorder. In some embodiments, the agent inhibits the antagonistic effect of the soluble assembly of amyloid β protein on NMDA receptor function. In some embodiments, NMDA receptor function is determined by whole cell patch clamp recording. In some embodiments, NMDA receptor function is determined by Ca⁺² fluorescence imaging. The present invention also includes agents identified by such methods.

The present invention includes a method of treating a neurodegenerative disease and/or a cognitive disorder in a subject, the method comprising administering to the subject an effective amount of an agent that alters the antagonistic effect of a soluble assembly of amyloid β protein on the NMDA receptor.

The present invention includes an isolated Aβ*56/NMDA receptor complex.

The present invention includes agents that alter the inhibitory effect of a soluble assembly of amyloid β protein on the NMDA receptor.

The present invention includes antibodies that bind to a soluble assembly of amyloid β protein and prevent the formation of an amyloid β/NMDA receptor complex. The present invention also includes methods of treating a neurodegenerative disease and/or a cognitive disorder in a subject, the method including administering to the subject an effective amount of such an antibody.

The present invention includes antibodies that bind to a NMDA receptor and prevent the formation of an amyloid β/NMDA receptor complex. The present invention also includes methods of treating a neurodegenerative disease and/or a cognitive disorder in a subject, the method including administering to the subject an effective amount of such an antibody.

In the methods of the present invention, the neurodegenerative disease and/or cognitive disorder may be Alzheimer's disease.

In any of the methods of the present invention, the soluble assembly of amyloid β protein may have a molecular weight of about 56 kDa as measured by SDS polyacrylamide gel electrophoresis.

In any of the methods of the present invention, the soluble assembly of amyloid β protein may be a dodecamer of amyloid β proteins.

In any of the methods of the present invention, the soluble assembly of amyloid β protein may be Aβ*56.

Unless otherwise specified, “a,” “an,” “the,” and “at least one” are used interchangeably and mean one or more than one.

### BRIEF DESCRIPTION OF THE FIGURES

FIGS. 1A-1C. Soluble Aβ*56 physically interacts with NMDA-receptor subunit NR1 and NR2A in Tg2576 brains. FIG. 1A shows Aβ*56 is present in both extracellular-enriched and membrane extracts. Western blots (WB) using anti-Aβ(1-16) antibodies (6E10) show multiples of Aβ trimers in addition to soluble APP (sAPPα) in soluble extracts of 13-month Tg2576 mice (left panel). In membrane extracts, full-length APP (fl-APP) and its BACE-generated CTF fragment, CTF-β, are detected along with Aβ monomers and Aβ*56 (right panel). Tg2576^{-/-} and Tg2576^{+/-} denote mice harbouring zero (non-Tg) and one transgene array, respectively; their ages (in months) are indicated in bold characters below the corresponding genotype. Synthetic human Aβ1-42 peptide (hAβ₄₂) was used as a size marker and positive control. Arrows indicate respective migration positions of monomers (1-mer), dimers (2-mer), trimers (3-mer), tetramers (4-mer), hexamers (6-mer), nonamers (9-mer) and dodecamers (12-mer), as well as sAPPβ and fl-APP. FIG. 1B shows Aβ*56 physically binds NR1 subunits in Tg2576 mice in an age-dependent manner. Immunoprecipitations (IP) using NR1 specific antibodies were used to capture potential Aβ*56-NMDA receptor complexes. NR1 receptor subunit levels after IP are shown in the lower panels as loading controls, and also to confirm the fidelity of the protein extraction protocol. FIG. 1C shows Aβ*56-NMDA receptor complexes are immunocaptured using anti-Aβ(17-24) antibodies (4G8). NR1 and NR2A, but not NR2B, complexes are readily pulled down and detected by both N-ter (N-terminus specific) and C-ter (C-terminus specific) NMDA receptor antibodies.

FIGS. 2A-2C. Human-derived Aβ*56 physically binds NMDA receptors. FIG. 2A shows Aβ*56 coimmunoprecipitates with NR1 NMDA receptor subunits in brain tissue from Alzheimer (AD) patients but not from control subjects with no cognitive impairment (NCI), or extracts containing no brain proteins (NP). FIG. 2B shows Aβ*56 co-immunoprecipitates with NR2A, but much less readily with NR2B, NMDA receptor subunits in brain tissue from subjects with AD but not from control subjects (NCI). FIG. 2C shows Aβ*56 does not coimmunoprecipitate with α nicotinic acetylcholine receptors (α7nAChR). Panels below each blot confirm the ability of the various receptor antibodies to immunoprecipitate the respective receptors or receptor subunits.

FIGS. 3A-3D. Aβ*56 increases calcium signaling and inhibits NMDA-evoked currents in cultured cortical neurons. FIG. 3A shows calcium fluorescence measured from a cluster of neurons. Addition of 7 nM Aβ*56 increases Ca²⁺ signaling in the cells, indicating a rise in circuit activity within the network of neurons in culture. FIG. 3B shows the Aβ*56-induced increase in Ca²⁺ signaling is blocked by an NMDA receptor antagonist (CPP) but not by an mGluR antagonist (E4CPG). Calcium signaling is not increased when the vehicle alone is added. Bars show means ±SD. FIG. 3C shows calcium fluorescence measured from a single neuron. Calcium signaling increases within seconds after addition of 7 nM Aβ*56 and remains raised for greater than 10 minutes following Aβ*56 washout. FIG. 3D shows the inward current evoked by focal ejection of 50 μM NMDA onto a neuron is reduced by addition of 1 nM Aβ*56.

FIGS. 4A-4B. Aβ*56 does not bind AMPA receptors. FIG. 4A shows antibodies to GluR1 and GluR2 subunits of AMPA receptors fail to immunoprecipitate Aβ*56 from soluble or membrane-enriched fractions generated from brains of 13-month Tg2576 mice. FIG. 4B shows GluR1 and GluR2 AMPA receptor subunits are immunoprecipitated by the GluR1 and GluR2 antibodies in the membrane-enriched but not the soluble fractions, excluding the possibility that the inability to detect Aβ*56 was due to failure of the antibodies to immunoprecipitate the AMPA receptor subunits. Tg2576^{-/-} and Tg2576^{+/-} denote mice harboring zero (non-Tg) and one transgene array, respectively. Synthetic human Aβ₁₋₄₂ peptide (hAP42) was loaded in parallel as a size marker and positive control. Arrows indicate respective migration positions of monomers (1-mer), dimers (2-mer), trimers (3-mer), tetramers (4-mer), hexamers (6-mer), nonamers(9-mer) and dodecamers (12-mer), as well as full-length APP (fl-APP) and soluble APP (sAPPα). Western blot (WB), immunoprecipitation (IP), soluble extracellular-enriched fraction (Sol), membrane-enriched fraction (MB).

FIG. 5. Similar levels of immunoglobulin G in immunoprecipitates from membrane enriched fractions indicate consistent loading between samples. Tg2576^{+/-} denotes mice harboring one transgene array; their ages (in months) are indicated above each gel. Arrows indicate the migration position of immunoglobulin G (IgG). Western blot (WB), immunoprecipitation (IP), Alzheimer brain (AD), brain from subjects with no cognitive impairment (NCI), no brain protein added (NP), no antibody added (No Ab).

FIGS. 6A-6B. Preparation of purified Aβ*56. FIG. 6A shows Tg2576 brain proteins from a 24-month mouse eluted after immunoaffinity purification in columns packed with 200 μg 6E10 (IPC 6E10-200) or 4G8 (IPC 4G8-200) antibodies, and probed with 6E10 antibodies by western blot (WB). FIG. 6B shoes optical density (A595) of fractions collected by size-exclusion chromatography of immunoaffinity-purified Tg2576 brain proteins using 4G8-packed columns. Silver stain of selected fractions corresponding to Aβ*56 (dodecamers) and AP trimers. Synthetic human Aβ₁₋₄₂ peptide (hAβ42) was loaded in parallel as a size marker and positive control. Arrows indicate respective migration positions of monomers (1-mer), dimers (2-mer), trimers (3-mer), tetramers (4-mer), hexamers (6-mer), nonamers (9-mer) and dodecamers (12-mer), as well as APP (combined full-length and soluble forms).

### DETAILED DESCRIPTION OF ILLUSTRATIVE EMBODIMENTS OF THE INVENTION

The present invention demonstrates for the first time that soluble assemblies of amyloid β (Aβ) protein bind to the NMDA receptor and function as an antagonist of the NMDA receptor. In particular, the present invention shows that Aβ*56, a soluble assembly of Aβ proteins, binds to NMDA receptors in both memory-impaired Tg2576 mice (a plaque-forming mice modeling Alzheimer disease) and patients with Alzheimer's disease (AD). The present invention also shows that Aβ*56 inhibits NMDA-evoked currents. NMDA receptors are critical mediators of long lasting synaptic plasticity and memory and the present invention defines a new mechanism by which Aβ proteins impair memory function in neurodegenerative disorders and cognitive disorders, such as Alzheimer's disease.

A major class of receptors for the neurotransmitter glutamate is referred to as N-methyl-D-aspartate receptors (NMDAR) since the receptor binds preferentially to N-methyl-D-aspartate (NMDA). NMDA is a chemical analog of aspartic acid. It normally does not occur in nature, and NMDA is not present in the brain. When molecules of NMDA contact neurons having NMDARs, they strongly activate the NMDAR (that is, they act as a powerful receptor agonist), causing the same type of neuronal excitation that glutamate does. The NMDA receptor is an excitatory, ionotropic receptor which plays a critical role in synaptic plasticity mechanisms and is necessary for several types of learning and memory. The NMDA receptor is a heteromeric, integral membrane protein formed by the assembly of obligatory NR1 subunits together with two modulatory NR2 subunits. The NR1 subunit is the glycine binding subunit and exists as 8 splice variants of a single gene. The glutamate binding subunit is the NR2 subunit, which is generated as the product of four distinct genes, and provides most of the structural basis for heterogeneity in NMDA receptors. A related gene family of NR3 A-C subunits can substitute for NR2 subunits in specific brain regions and has an inhibitory effect on receptor activity. Multiple receptor isoforms with distinct brain distributions and functional properties arise by selective splicing of the NR1 transcripts and differential expression of the NR2 subunits. See, for example, Dingledine et al., “The glutamate receptor ion channels,” Pharmacol Rev. 1999 51(1):7-61. As used herein, a NMDA receptor has an NR1 subunit and at least one of four different NR2 and NR3 subunits (designated as NR2A, NR2B, NR2C, and NR2D, NR3A and NR3B). An exemplary NR1 subunit is the human NMDAR1 polypeptide. The sequence of the polypeptide and corresponding nucleic acid may be obtained at Genbank, accession number L05666, and is published in Planells-Cases et al. (1993) P.N.A.S. 90(11):5057-5061. An exemplary NR2 subunit is the human NMDAR2A polypeptide. The sequence of the polypeptide and corresponding nlucleic acid may be obtained at Genbank, accession number U09002, and is published in Foldes et al. (1994) Biochim. Biophys. Acta 1223 (1):155-159. Another NR2 subunit is the human NMDAR2B polypeptide. The sequence of the polypeptide and corresponding nucleic acid may be obtained at Genbank, accession number U11287, and is published in Adams et al. (1995) Biochim. Biophys. Acta 1260 (1):105-108. NMDA receptors are “ionotropic” receptors since they flux ions, such as Ca²⁺. These ion channels allow ions to flow into a neuron upon depolarization of the postsynaptic membrane, when the receptor is activated by glutamate, aspartate, or an agonist drug.

As used herein an “amyloid-beta protein,” also referred to as an “amyloid-beta polypeptide,” an “amyloid-beta peptide,” an “amyloid-beta molecule,” an “amyloid-β protein,” an “amyloid-β polypeptide,” an “amyloid-β peptide,” and “amyloid-β molecule,” an “Aβ protein,” an “Aβ polypeptide,” an “Aβ peptide,” an “Aβ molecule,” “amyloid-beta,” “amyloid β,” or “Aβ,” is the major constituent of amyloid plaques in the brains of individuals afflicted with Alzheimer's disease, a polypeptide of 39 to 43 amino acid residue first identified by Glenner and Wong (see, for example, Glenner et al., (1984) Biochem Biophys Res Commun 120, 885-890; and Glenner and Wong (1984) Biochem Biophys Res Commun 122, 1131-1135) and Masters et al. (See Masters et al., (1985) Embo J 4, 2757-2764; and Masters et al., (1985) Proc Natl Acad Sci USA 82, 4245-4249). The gene for the amyloid precursor protein (APP) of the amyloid-beta protein has been cloned and sequenced (see, for example, Kang et al., (1987) Nature 325, 733; Tanzi et al., (1987) Science 235, 880-884; and Selkoe (1994) Annual Review of Neuroscience Vol, 17, 489-517). As used herein, an amyloid-beta protein may be any of the various known allelic variants and mutations of the amyloid-beta protein.

Amyloid beta peptide is generated from the beta-amyloid precursor protein (beta APP) in a two-step process. The first step involves cleavage of the extracellular, amino-terminal domain of beta APP. Protein cleavage is performed by an aspartyl protease termed beta-secretase (BACE). This enzyme is synthesized as a propeptide that must be modified to the mature and active form by the prohormone convertase, furin. Beta APP cleavage by the mature form of BACE results in the cellular secretion of a segment of beta APP and a membrane-bound remnant. This remnant is then processed by another protease termed gamma-secretase. Gamma-secretase cleaves an intra-membrane site in the carboxyl-terminal domain of beta APP, thus generating the amyloid beta peptide. Gamma-secretase is believed to be a multi-subunit complex containing presenilin-1 and 2 as central components. Found associated with the presenilins is the transmembrane glycoprotein nicastrin. Nicastrin has been found to bind to the carboxyl-terminus of betaAPP and helps to modulate the production of the amyloid beta peptide. Also found in the neurofibrillary lesions in Alzheimer's disease is the protein termed Tau. Tau is a neuronal microtubule-associated protein found predominantly on axons. The function of tau is to promote tubulin polymerization and stabilize microtubules. Tau, in its hyperphosphorylated form, is the major component of paired helical filaments (PHF), which is the building block of neurofibrillary lesions in Alzheimer's disease brain. See, for example, J. Neurosci. 18:1743-1752, 1998 and Neuron, 19:939-945, 1997.

As used herein, an amyloid-beta protein is a monomeric polypeptide, made up of one polypeptide chain. A monomeric polypeptide is also referred to herein as a “monomer.”

As used herein, an oligomer of amyloid β, also referred to an oligomeric form of amyloid β, is a detergent-stable configuration of more than one amyloid-beta protein. An oligomer is not necessarily polymerized. An oligomer of amyloid β may be soluble. As used herein a “dimer” is a detergent-stable configuration of two amyloid-beta proteins. As used herein a “trimer” is a detergent-stable configuration of three amyloid-beta proteins. As used herein a “tetramer” is a detergent-stable configuration of four amyloid-beta proteins. As used herein a “pentamer” is a detergent-stable configuration of five amyloid-beta proteins. As used herein a “hexamer” is a detergent-stable configuration of six amyloid-beta proteins.

As used herein, an “assembly” is a configuration of one or more oligomers of Aβ proteins. In a preferred embodiment, an assembly is a configuration of more than one Aβ protein oligomer. An assembly of oligomers of Aβ proteins may be, for example, an assembly of two oligomers of Aβ proteins, three oligomers of Aβ proteins, four oligomers of Aβ proteins, five oligomers of Aβ proteins, six oligomers of AP proteins, or more oligomers of Aβ proteins. In some embodiments, an assembly of oligomers of Aβ proteins may be, for example, a nanomer of nine amyloid β proteins or a dodecamer of twelve amyloid β proteins. In some embodiments, an assembly of oligomers of Aβ proteins may be, for example, an assembly of more than one hexamer of amyloid β proteins, more than one pentamer of amyloid β proteins, more than one tetramer of amyloid β proteins, more than one trimer of amyloid β proteins, or more than one dimer of amyloid β proteins. In some embodiments, an assembly of oligomers of Aβ proteins may be, for example, an assembly of two hexamers of amyloid β proteins, three hexamers of amyloid β proteins, two tetramers of amyloid β proteins, three tetramers of amyloid β proteins, four tetramers of amyloid β proteins, two trimers amyloid β proteins, three trimers amyloid β proteins, four trimers of amyloid β proteins, five trimers amyloid β proteins, two dimers of amyloid β proteins, three dimers of amyloid β proteins, four dimers of amyloid β proteins, five dimers of amyloid β proteins, six dimers of amyloid β proteins, seven dimers of amyloid β proteins, or eight dimers of amyloid β proteins.

In some embodiments, amyloid-β protein assemblies may include detergent-stable dimers of amyloid-β protein. In some embodiments, amyloid-β protein assemblies may include detergent-stable trimers of amyloid-β protein. In some embodiments, amyloid-β protein assemblies may include detergent-stable tetramers of amyloid-β protein. In some embodiments, amyloid-β protein assemblies may include detergent-stable pentamers of amyloid-β protein. In some embodiments, amyloid-β protein assemblies may include detergent-stable hexamers of amyloid-β protein.

The present invention also includes isolated, soluble amyloid-,B protein assemblies having one or more amyloid-β protein trimers. As used herein an “amyloid-β protein trimer” is a detergent-stable configuration of three Aβ molecules. In some embodiments, a soluble amyloid-β protein assembly has more than one amyloid-β protein trimer. In some embodiments, the amyloid-β protein assembly includes three amyloid-β protein trimers. In some embodiments, the amyloid-β protein assembly is a nonamer of amyloid-β proteins. In some embodiments, an amyloid-β protein assembly has a molecular weight of about 40 kDa as measured by SDS polyacrylamide gel electrophoresis. In some embodiments, the amyloid-β protein assembly includes four amyloid-β protein trimers. In some embodiments, the amyloid-β protein assembly has a molecular weight of about 56 kDa as measured by SDS polyacrylamide gel electrophoresis.

In some embodiments, amyloid-β protein assemblies may be a dodecamer of amyloid-β proteins. Such dodecamers of amyloid-β proteins may be six dimers of amyloid-β protein, four trimers of amyloid-β protein, three tetramers of amyloid-β protein, or two hexamers of amyloid-β protein. In some embodiments, the dodecamer of amyloid-β proteins has a molecular weight of about 56 kDa as measured by SDS polyacrylamide gel electrophoresis.

As used herein, a detergent-stable, also referred to herein as “detergent stable,” configuration does not disassemble or disassociate into its component subunits in a detergent solution. Such a detergent solution may be, for example, a 1% solution Triton X-100 or a 2% solution of SDS. Thus, a detergent stable oligomer of amyloid-β protein does not disassociate into separate amyloid-β protein monomers in a detergent solution.

The assemblies of amyloid β protein of the present invention are soluble. As used herein, the term “soluble” means remaining in aqueous solution. In some embodiments, soluble assemblies of amyloid β protein remain in the supernatant after centrifugation, including, for example, ultracentrifugation. Soluble assemblies of amyloid β protein may remain in solution in a wide range of solutions, including, but not limited to, water, in an isotonic solution, tissue culture medium, a buffered solution, a detergent buffer, an organic buffer, or a body fluid, including, for example, plasma or cerebrospinal fluid. Assemblies of amyloid β protein may remain in solution in a physiological buffer.

Assemblies of amyloid β protein may remain in solution in range of temperatures. For example, the assemblies of amyloid β protein may remain in solution at a temperature greater than 0° C. Assemblies of amyloid β protein may remain in solution, for example, at a temperature of at least about 4° C., at a temperature of at least about 10° C., at a temperature of at least about 15° C., at a temperature of at least about 25° C., at a temperature of at least about 37° C., at a temperature of at least about 42° C., at a temperature of at least about 50° C., at a temperature of at least about 55° C., at a temperature of at least about 60° C., at a temperature of at least about 70° C., at a temperature of at least about 75° C., at a temperature of at least about 80° C., at a temperature of at least about 85° C., at a temperature of at least about 90° C., at a temperature of at least about and/or at a temperature of at least about 95° C.

Assemblies of amyloid β protein may remain in solution, for example, at a temperature of less than about 4° C., at a temperature of less than about 10° C., at a temperature of less than about 15° C., at a temperature of less than about 25° C., at a temperature of less than about 37° C., at a temperature of less than about 42° C., at a temperature of less than about 50° C., at a temperature of less than about 55° C., at a temperature of less than about 60° C., at a temperature of less than about 70° C., at a temperature of less than about 75° C., at a temperature of less than about 80° C., at a temperature of less than about 85° C., at a temperature of less about 90° C., at a temperature of less than about 95° C., and/or at a temperature of less than about 100° C.

Assemblies of amyloid β protein may remain in solution, for example, at a temperature of about 4° C., at a temperature of about 10° C., at a temperature of about 15° C., at a temperature about 25° C., at a temperature of about 37° C., at a temperature of about 42° C., at a temperature of at about 50° C., at a temperature of about 55° C., at a temperature of about 60° C., at a temperature of about 70° C., at a temperature of about 75° C., at a temperature of at about 80° C., at a temperature of about 85° C., at a temperature of about 90° C., and/or at a temperature of about 95° C.

Assemblies of amyloid β protein may remain in solution in a range of any of the various temperatures discussed above.

Soluble assemblies of AB include isolated, soluble, non-fibrillar amyloid-β protein (Aβ) assemblies having one or more detergent-stable oligomers of amyloid-β protein. The soluble, non-fibrillar amyloid-β protein (Aβ) assemblies of the present invention may be made up of one or more detergent-stable oligomers of amyloid-β protein. The soluble, non-fibrillar amyloid-β protein (Aβ) assemblies of the present invention may also be referred to herein as Aβ*assemblies, Aβ*molecules, AP star assemblies, Aβ star molecules, A-beta*assemblies, A-beta*molecules, A-beta star assemblies, A-beta star molecules, Aβ*56, or A*56 (PCT/US2005/037828, “Assemblies of Oligomeric Ainyloid Beta Protein and Uses Thereof” and Lesne et al., Nature. 2006 Mar 16;440(7082):352-7, “A specific amyloid-beta protein assembly in the brain impairs memory”). In some embodiments, a soluble, non-fibrillar amyloid-β protein (Aβ) assembly has more than one detergent-stable oligomers of amyloid-β protein. In some embodiments, the soluble, non-fibrillar amyloid-β protein (Aβ) assemblies of the present invention may be isolated and purified.

Assemblies of amyloid β protein may be obtained from a wide variety of sources. Assemblies of amyloid β protein may be obtained from natural sources; for example, from natural fluids, cells, or tissues, including, but not limited to, plasma, brain tissue, and cerebrospinal fluid. Assemblies of amyloid β may be isolated from the culture medium of cells expressing endogenous or transfected amyloid β protein precursor genes. For example, assemblies of oligomers of amyloid β protein may be obtained from the culture medium of Chinese hamster ovary (CHO) cells stably transfected to express amyloid β protein (Podlinsky et al., J Biol. Chem., 1995, 270(16):9564-9570). Assemblies of amyloid β protein may be synthetically produced. Assemblies of amyloid β protein may be produced recombinantly.

Assemblies of amyloid-β protein disrupt cognitive functioning, representative of a cognitive disorder. Such cognitive disorders include, but are not limited to, mild cognitive impairment, memory deficits, age related memory decline, age associated memory impairment, and Alzheimer's disease, including, but not limited to presymptomatic Alzheimer's disease and early Alzheimer's disease. Disruptions of cognitive function may be representative of any phase of a neurological disorder, including, but not limited to, a presymptomatic phase, a preclinical phase, or an early phase of a neurological disorder. The disruption of cognitive function may be representative of age-related memory decline or age-associated memory impairment (see Craik, F. I. in Handbook of the Psychology of Aging (eds. Birren, J. E. & Schall, K.) 384-420 (Van Nostrand-Reinhold, New York, 1977) and Morrison and Hof, Science 1997, 278;412-9). Such functional deficiencies may be transient or permanent. Such functional deficiencies may be observed in the absence of neuropathological damage. Such neuropathologies may include, for example, amyloid plaque formation, amyloid deposits, oxidative stress, astrogliosis, microgliosis, cytokine production, dystrophic neurons, formation of neurobifillary tangles, neurodegeneration, gross neuronal atrophy, neuronal loss, synaptic loss, and other manifestations of neuropathology.

Methods for assaying disruption of learned behavior and/or cognitive functioning can include, for example, those described in Cleary et al., Nat. Neuroscience 8, 79-84 (2005); U.S. Provisional Application 60/584,695 (filed Jun. 30, 2004); U.S. Provisional Application 60/621,549 (filed Oct. 22, 2004); and PCT Application “Soluble Oligomers of Amyloid Beta Disrupt Memory of Learned Behavior” (filed Jun. 30, 2005).

Cognitive disruption may be assayed by any of a variety of methods. One means of assessing cognitive functioning is the Alternating Lever Cyclic Ratio (ALCR) test, which has proven to be sensitive for measuring cognitive function (O'Hare et al., Behav Pharmacol 1996, 7:742-753; and Richardson et al., Brain Res 2002, 954:1). Under ALCR, rats learn a complex sequence of lever-pressing requirements for food reinforcement in a two-lever experimental chamber. Rats must alternate between the two levers, switching to the other lever after pressing the first lever enough to get a food pellet. The number of presses required for each food reward proceeds from low (2 presses) to high (56 presses), incorporating intermediate values based on the quadratic function, x²−x. One cycle is an entire ascending and descending sequence of these response requirements (for example, 2, 6, 12, 20, 30, 42, 56, 56, 42, 30, 20, 12, 6, and 2 presses per food reward). Six such full cycles are presented during each session. Errors are scored when the subject perseveres on a lever after reward, that is, does not alternate (a perseveration error), or when a subject switches levers before completing the response requirement on that lever (a switching error).

Other procedures that may be used to assess cognitive functioning, include, but are not limited to, a delayed non-matching to place test, a morris water maze (commonly used to assess working memory in rats and mice), a delayed matching to sample test (an operant procedure for testing working memory), and a fixed-interval operant responding test (a sensitive procedure to assess non-specific cognitive effects, for example, when the type and anatomical location of the cognition being tested is unknown), a delayed conditioning procedure (representing a variety of operant or non-operant tests under which animals are exposed to stimuli paired with a reward or punishment and, after a delay, their ability to respond appropriately to the stimulus-reward combination is assessed), or a repeated acquisition procedure (an operant test, under which subjects are required to repeatedly learn a new stimulus sequence).

The present invention includes a method for detecting the presence of assemblies of amyloid β protein in a sample taken from a subject by contacting a sample with an antibody to the NMDA receptor, a NMDA receptor subunit and/or an antibody to a complex of the NMDA receptor and soluble assemblies of amyloid β and detecting binding of the antibody. The sample may be, for example, serum, blood, cerebrospinal fluid (CSF), or brain tissue.

The present invention includes a method of detecting a neurodegenerative disease and/or cognitive disorder in a subject, the method including obtaining a sample from the subject; immunoprecipitating the sample with an antibody to a NMDA receptor or receptor subunit; wherein the coprecipitation of amyloid β along with the NMDA receptor indicates the subject has a neurodegenerative disease and/or cognitive disorder. In some aspect, the amyloid β is a soluble assembly of amyloid β protein, including any of the soluble assemblies of amyloid β described herein, including, but not limited to, Aβ*56. In some aspects, the antibody to an NMDA receptor is an antibody that binds to a NMDA receptor subunit selected from NR1, NR2A, and/or NR2B.

The present invention also includes a method of detecting a presymptomatic neurodegenerative disease and/or cognitive disorder in a subject, the method including obtaining a sample from the subject; immunoprecipitating the sample with an antibody to a NMDA receptor or receptor subunit; wherein the coprecipitation of amyloid β along with the NMDA receptor indicates the subject has a presymptomatic neurodegenerative disease and/or cognitive disorder disease. In some embodiments, the amyloid β is a soluble assembly of amyloid β protein, including any of the soluble assemblies of amyloid β described herein, including, but not limited to, Aβ*56. In some aspects, the antibody to an NMDA receptor is an antibody that binds to a NMDA receptor subunit selected from NR1, NR2A, and/or NR2B. The method may be used for detecting Alzheimer's disease. The method may be used for detecting presymptomatic Alzheimer's disease.

The present invention includes a method of inhibiting NMDA receptor function, the method including contacting a NMDA receptor with a soluble assembly of amyloid β protein, including any of the soluble assemblies of amyloid β described herein. In some embodiments, the soluble assembly of amyloid β protein is Aβ*56.

The present invention includes methods of screening for agents that alter or modulate the antagonistic effect of soluble assemblies of amyloid β on NMDA receptor function. Such a method may include contacting a NMDA receptor with both an agent and a soluble assembly of amyloid β, determining NMDA receptor function for the NMDA receptor contacted with both the agent and the soluble assembly of amyloid β, comparing NMDA receptor function for the NMDA receptor contacted with both the agent and the soluble assembly amyloid β to NMDA receptor function for a NMDA receptor contacted with the soluble assembly of amyloid β and not contacted with the agent. A difference in the level of NMDA receptor function in the NMDA receptor contacted with both the agent and the soluble assembly of amyloid β compared to NMDA receptor function in the NMDA receptor contacted with the soluble assembly of amyloid β and not contacted with the agent indicates the agent alters the antagonistic effect of the soluble assembly of amyloid β on NMDA receptor function. A soluble assembly of amyloid β can include any of those described herein, including, but not limited to, Aβ*56.

As used herein, an antagonistic effect is an inhibition or decrease in the normal physiological function of a receptor. An antagonist that competes with an agonist for a receptor is a competitive antagonist. An antagonist that antagonize by other means is a non-competitive antagonist.

The present invention includes methods of screening for agents effective for the treatment of neurodegenerative diseases and/or cognitive disorders. Such a method may include contacting a NMDA receptor with an agent and a soluble assembly of amyloid β, determining NMDA receptor function for the NMDA receptor contacted with the agent and the soluble assembly of amyloid β, comparing NMDA receptor function for the NMDA receptor contacted with the agent and the soluble assembly of amyloid β to NMDA receptor function for an NMDA receptor contacted with the soluble assembly of amyloid β and not contacted with the agent. An altered level of NMDA receptor function in the NMDA receptor contacted with the agent and the soluble assembly of amyloid β compared to the NMDA receptor function of the NMDA receptor contacted with the soluble assembly of amyloid β and not contacted with the agent indicates the agent may be effective for the treatment of neurodegenerative disease and/or cognitive disorder. A soluble assembly of amyloid p can include any of those described herein, including, but not limited to, Aβ*56. The present invention also includes agents identified by the screening methods described herein and methods of treatment that include the administration of such agents.

NMDA receptors used in such methods may be provided in any of a wide variety of formats, including for example, as isolated receptors, reconstituted membranes, cell membrane preparations, whole cells, or tissues. For example, neuronal cell cultures, neurocortical cell cultures, and nerve and brain tissues. NMDA receptor function may be determined by any of a wide variety of means. For example, NMDA receptor function may be determined by assaying for changes in concentrations of intracellular calcium in cells or tissues maintained in tissue culture. Calcium concentration may be determined, for example, by the use of a calcium indicator dye.

Calcium indicator dyes (also called calcium ion probes) are widely used intracellular indicators (Cellular Calcium, A Practical Approach, (1991) McCormack, J. G. and P. H. Cobbold eds. IRL Press at Oxford Press, New York, N.Y.). Calcium ion detection may be accomplished by using a dye that has a recognition portion as well as a region that confers fluorescence. One commonly used structure for calcium specific binding is 1,2-bis(2-aminophenoxy)ethane-N,N,N′,N′-tetraacetic acid, (BAPTA). Calcium indicator dyes can be categorized into at least two groups; the first are the dyes that increase their fluorescence in the presence of calcium, while the second group are dyes that have different excitation and/or emission wavelengths in the presence of calcium than they have in its absence. The calcium indicator dyes, calcium green-1, calcium green-2, and Fluo-4 are representative of the dyes that increase their fluorescence in the presence of calcium ion without changing wavelengths. Fura-2 and Indo-1 are ratiometric Ca²⁺ indicators that are generally considered interchangeable in most experiments. Fura-2, upon binding Ca2+, exhibits a shift in its absorption or excitation peak from 338 nm to 366 nm (Haughland, R., (2002) Handbook of Fluorescent Compounds and Research Products, ninth Ed., Molecular Probes, Inc), making Fura-2 the dye a common choice for microscopy, where it is easier to change excitation wavelengths than emission. Indo-1 on the other hand has a shift in the emission from 485 nm to 405 nm in the presence of calcium. Thus, Indo-1 has a greater utility with flow cytometry where it is easier to use a single argon-ion laser for excitation and to monitor two different emissions. Calcium indicator dyes can be efficiently measured using filter based microplate readers. See, also, Principles of Fluorescence Spectroscopy 2nd Edition (1999) Lakowicz, J. R. Editor, Kluwer Academic/Plenum Publishers, New York, N.Y.; The Encyclopedia of Molecular Biology (1994) Kendrew, J Editor, Blackwell Science Ltd. Cambridge, Mass.; Cellular Calcium, A Practical Approach, (1991) McCormack, J. G. and P. H. Cobbold eds. IRL Press at Oxford Press, New York, N.Y.; Haughland, R., (2002) Handbook of Fluorescent Compounds and Research Products, ninth Ed., Molecular Probes, Inc; and P. Held, (Jun. 6, 2003) “Detection of Calcium Concentration Changes Using the FLx800 Fluorescence Microplate Reader,” Applications Department, Bio-Tek Instruments, Inc., available on the world wide web at biotek.com/resources/tech_res_detail.php?id.

Alternatively, NMDA receptor function may be determined in whole cell patch clamp assays, as described in more detail herein. Alternatives to whole cell patch clamp assays may be employed, including, for example, discontinuous single electrode voltage-clamp (dSEVC) (Roelfsema et al., J Exp Bot. 2001 September;52(362):1933-9) and high-throughput methods utilizing multielectrode extracellular recordings of cell-electrode hybrids (Nataraj an et al., Toxicol In Vitro. 2006 April;20(3):375-81. Epub 2005 Sept. 29).

Agents of the present invention alter the antagonistic effect of a soluble assembly of amyloid β on NMDA receptor function. Altering an antagonistic effect includes inhibiting or decreasing the antagonistic effect. Altering an antagonistic effect includes increasing or enhancing the antagonistic effect. As used herein, an “agonist” or “activator” is a molecule which, when interacting with a target receptor protein prolongs the amount or duration of the effect of the biological activity of the target protein. By contrast, the term “antagonist,” or “inhibitor” as used herein, refers to a molecule which, when interacting with a target protein, decreases the amount or the duration of the effect of the biological activity of the target protein. Agonists and antagonists include, but are not limited to, proteins, nucleic acids, carbohydrates, antibodies, or any other molecules that decrease the effect of a protein.

The term “analog” is used herein to refer to a molecule that structurally resembles a molecule of interest but which has been modified in a targeted and controlled manner, by replacing a specific substituent of the reference molecule with an alternate substituent. Compared to the starting molecule, an analog may exhibit the same, similar, or improved utility. Synthesis and screening of analogs, to identify variants of known compounds having improved traits (such as higher potency at a specific receptor type, or higher selectivity at a targeted receptor type and lower activity levels at other receptor types) is an approach that is well known in pharmaceutical chemistry. The present invention includes analogs of the agents described herein.

The terms “modulation” or “alteration,” as used herein, refer to both up regulation, (activation or stimulation), for example by agonizing; and down regulation (i.e. inhibition or suppression), for example by antagonizing, of a bioactivity. Modulators include, but are not limited to, both “activators” and “inhibitors” of function. An “activator” is a substance that directly or indirectly enhances function, causing the NMDA receptor to become more active. Conversely, an “inhibitor” directly or indirectly decreases NMDA receptor function, causing the NMDA receptor to become less active. The reduction may be complete or partial. As used herein, modulators of NMDA-R signaling encompass antagonists and agonists.

In some embodiments, the ability of an agent to enhance or inhibit NMDA-R activity is assayed in an in vitro system. In general, the in vitro assay format involves adding an agent and NMDA-R, and measuring the biological activity of the NMDA receptor.

In the assays and methods of the present invention, receptor function of NMDA receptors contacted with both an agent and a soluble assembly of amyloid β may be compared to receptor function of NMDA receptors contacted with only a soluble assembly of amyloid β and not contacted with the agent. NMDA receptor function of NMDA receptors contacted with only the soluble assembly of amyloid β and not contacted with the agent may be determined within the assay, by contacting NMDA receptors with the soluble assembly of amyloid β and determining receptor function. Alternatively, receptor function of NMDA receptors contacted with only the soluble assembly of amyloid β and not contacted with the agent may be provided as a value

The present invention includes antibodies that bind to a soluble assembly of amyloid β protein and prevent the formation of an amyloid β/NMDA receptor complex. Such antibodies may be used in methods of detecting and treating a neurodegenerative disease and/or a cognitive disorder in a subject, the method including administering to the subject an effective amount of such an antibody.

The present invention includes antibodies that bind to a NMDA receptor and prevent the formation of an amyloid β/NMDA receptor complex. Such antibodies may be used in methods of detecting and treating a neurodegenerative disease and/or a cognitive disorder in a subject, the method including administering to the subject an effective amount of such an antibody.

The present invention includes antibodies that bind to an amyloid β/NMDA receptor complex NMDA receptor but do not bind to isolated amyloid β and do not bind to an isolated NMDA. Such antibodies may be used in methods of detecting and treating a neurodegenerative disease and/or a cognitive disorder in a subject, the method including administering to the subject an effective amount of such an antibody.

The antibodies of the present invention can be produced and characterized by any of many methods, including, but not limited to, any of the methods described herein. The ability of an antibody to inhibit the Aβ*56-mediated inhibition of NMDA-evoked currents may be determined by any of many available assays, including, but not limited to, any of the assays described herein.

Also included in the present invention are compositions including one or more of the antibodies as described herein. Such compositions may include a pharmaceutically acceptable carrier. Also included in the present invention are kits with one or more of the antibodies of the present invention.

As used herein, the terms “antibody” or “antibodies” includes polyclonal antibodies, affinity-purified polyclonal antibodies, monoclonal antibodies, and antigen-binding fragments thereof, such as F(ab′)₂ and Fab proteolytic fragments. Genetically engineered intact antibodies or fragments, such as chimeric antibodies, Fv fragments, single chain antibodies and the like, as well as synthetic antigen-binding peptides and polypeptides, are also included. The term “polyclonal antibody” refers to an antibody produced from more than a single clone of plasma cells; in contrast “monoclonal antibody” refers to an antibody produced from a single clone of plasma cells. Polyclonal antibodies may be obtained by immunizing a variety of warm-blooded animals such as horses, cows, goats, sheep, dogs, chickens, rabbits, mice, hamsters, guinea pigs and rats as well as transgenic animals such as transgenic sheep, cows, goats or pigs, with an immunogen. Monoclonal antibodies can be obtained by various techniques familiar to those skilled in the art.

A therapeutically useful antibody may be derived from a “humanized” monoclonal antibody. Humanized monoclonal antibodies are produced by transferring one or more CDRs from the heavy and light variable chains of a mouse (or other species) immunoglobulin into a human variable domain, then substituting human residues into the framework regions of the murine counterparts. The use of antibody components derived from humanized monoclonal antibodies obviates potential problems associated with immunogenicity of murine constant regions. Techniques for producing humanized monoclonal antibodies can be found, for example, in Jones et al., Nature (1986);321: 522 and Singer et al., J. Immunol., (1993);150: 2844.

Antibodies of the present invention may, for example, be administered following any of the procedures used for the administration of the antibodies to tumor necrosis fact (TNF) adalimumab (also known as HUMIRA) (see, for example, http://www.rxabbott.con/pdf/ humira.pdf or Baker, DE, “Adalimumab: human recombinant immunoglobulin G1 anti-tumor necrosis factor monoclonal antibody,” Rev Gastroenterol Disord. 2004 Fall;4(4):196-210) or infliximab (also known as REMICADE) (see, for example, http://www.remicade.com/pdf/IN04810.pdf, Harriman et al., “Summary of clinical trials in rheumatoid arthritis using infliximab, an anti-TNFalpha treatment,” Ann Rheum Dis. 1999 November;58 Suppl 1:I61-4, or Hochberg et al., “Comparison of the efficacy of the tumour necrosis factor alpha blocking agents adalimumab, etanercept, and infliximab when added to methotrexate in patients with active rheumatoid arthritis,” Ann Rheum Dis. 2003 November;62 Suppl 2:ii13-6).

In addition, chimeric antibodies can be obtained by splicing the genes from a mouse antibody molecule with appropriate antigen specificity together with genes from a human antibody molecule of appropriate biological specificity; see, for example, Takeda et al., Nature (1985);314: 544-546. A chimeric antibody is one in which different portions are derived from different animal species.

The phrase “specifically binds” or “specifically immunoreactive with,” when referring to an antibody, refers to a binding reaction that is determinative of the presence of a protein in a heterogeneous population of proteins and other biologics. Thus, under designated immunoassay conditions, the specified antibodies bind to a particular protein at least about two times the background and do not substantially bind in a significant amount to other proteins present in the sample. Typically a specific or selective reaction will be at least about twice background signal or noise and more typically more than about 10 to about 100 times background. Specific binding to an antibody under such conditions may require an antibody that is selected for its specificity for a particular protein.

“Binding affinity” or “affinity binding” refers to the strength of the sum total of noncovalent interactions between a single binding site of a molecule (e.g., an antibody) and its binding partner (e.g., an antigen or antigenic epitope). The affinity of a molecule X for its partner Y is represented by the dissociation constant (Kd), which can generally be determined by using methods known in the art, for example, using the BIAcore biosensor, commercially available from BIAcore Inc., Piscataway, N.J. Antibodies of the present invention can also be described in terms of their binding affinity for the amyloid β, Aβ*56, the NMDS receptor and/or a complex of the NMDA receptor and amyloid β.

Antibodies of the present invention can be assayed for specific binding by any suitable method known in the art. The immunoassays that can be used include but are not limited to competitive and non-competitive assay systems using techniques such as BIAcore analysis, FACS (Fluorescence activated cell sorter) analysis, immunofluorescence, immuno cyto chemistry, Western blots, radio-immuno assays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see e.g., Ausubel et al, eds, Current Protocols in Molecular Biology, Vol. 1, John Wiley & Sons, Inc., N.Y. (1994)).

The present invention includes hybridoma cell lines, transformed B-cell lines, host cells, and progeny, derivatives or equivalents thereof producing the antibodies of the present invention. The present invention also includes polynucleotides encoding an antibody of the present invention, or antigen-binding fragment thereof.

Various delivery systems are known and can be used to administer the agents, antibodies or pharmaceutical compositions of the invention. Methods of introduction include but are not limited to intradermal, intramuscular, intraperitoneal, intravenous, subcutaneous, intranasal, epidural, and oral routes. The compounds or compositions can be administered by any convenient route, for example, by infusion or bolus injection, by absorption through epithelial or mucocutaneous linings (e.g., oral mucosa, rectal and intestinal mucosa, etc.) and can be administered together with other biologically active agents. Administration can be systemic or local. In some embodiments, it can be desirable to administer the pharmaceutical compounds or compositions of the invention locally to the area in need of treatment; this can be achieved, for example, by local infusion during surgery, by topical application, in conjunction with a wound dressing after surgery, by injection, by means of a catheter, by means of a suppository, or by means of an implant. In other embodiments, the compound or composition can be delivered in a vesicle, for example, a liposome. In yet other embodiments, the compound or composition can be delivered in a controlled release system. In some embodiments a pump can be used. In other embodiments polymeric materials can be used.

The present invention also provides pharmaceutical compositions. Such compositions comprise a therapeutically effective amount of compound of the invention, and a pharmaceutically acceptable carrier. In some embodiments, the term “pharmaceutically acceptable” means approved by a regulatory agency of the Federal or a state government or listed in the U.S. Pharmacopeia or other generally recognized international pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. In some embodiments, water can be used as a carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsions, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, or magnesium carbonate. Examples of suitable pharmaceutical carriers are described in Remington's Pharmaceutical Sciences by E. W. Martin. Such compositions will contain a therapeutically effective amount of the compound, preferably in purified form, together with a suitable amount of carrier so as to provide the form for proper administration to the patient. The formulation should suit the mode of administration.

In other embodiments, the composition is formulated in accordance with routine procedures as a pharmaceutical composition adapted for intravenous administration to a subject. Typically, compositions for intravenous administration are solutions in sterile isotonic aqueous buffer. Where necessary, the composition can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Wliere the composition is administered by injection, an ampoule of sterile water for injection or saline can be provided so that the ingredients can be mixed prior to administration.

The agents and/or antibodies of the present invention can be used either alone or in combination with other compounds or compositions. The agents and/or antibodies of the present invention can be used in both in vitro and in vivo diagnostic and therapeutic methods. Also included in the present invention are such in vitro and in vivo diagnostic and therapeutic methods.

An agent or antibody may be administered by any of a wide variety of means. For example, delivered orally, subcutaneaously, intramuscularly, intravenously, intrathecally, and/or intracranially. Delivery may be by local delivery or injection. Delivery may be by pump or extended release composition. An agent or antibody may be delivered prior to, during, and/or after delivery of another therapeutic agent. One or more agents may be administered.

The invention also provides a kit including the agents and/or antibodies of the present invention. The kit can include one or more containers filled with one or more of the agents and/or antibodies of the invention. Additionally, the kit may include other reagents such as buffers and solutions needed to practice the invention are also included. Optionally associated with such container(s) can be a notice or printed instructions. As used herein, the phrase “packaging material” refers to one or more physical structures used to house the contents of the kit. The packaging material is constructed by well known methods, preferably to provide a sterile, contaminant-free environment. As used herein, the term “package” refers to a solid matrix or material such as glass, plastic, paper, foil, and the like, capable of holding within fixed limits a polypeptide. Thus, for example, a package can be a glass vial used to contain milligram quantities of an antibody.

The present invention also includes agents identified by the screening methods described herein and methods of treatment that include the administration of such agents. Such agents may be administered to a subject for the treatment of a neurodegenerative disease and/or cognitive disorder, including, but not limited to Alzheimer's disease. Suitable agents include any of a wide variety of molecules. The term “agent” includes any substance, molecule, element, compound, entity, or a combination thereof. It includes, but is not limited to, e.g., polypeptides, oligopeptide, small organic molecule, polysaccharide, polynucleotide, antisense molecules, ribozymes, antibodies, and the like. It can be a natural product, a synthetic compound, or a chemical compound, or a combination of two or more substances. Unless otherwise specified, the terms “agent,” “substance,” and “compound” can be used interchangeably.

As used herein “treating” or “treatment” includes both therapeutic and prophylactic treatments. Desirable effects of treatment include preventing occurrence or recurrence of disease, alleviation of symptoms, diminishment of any direct or indirect pathological consequences of the disease, decreasing the rate of disease progression, amelioration or palliation of the disease state, and remission or improved prognosis.

The present invention includes an isolated complex of soluble assemblies of amyloid β and the NMDA receptor. In a preferred embodiment, the isolated complex is a complex between a NMDA receptor and Aβ*56. Isolated complexes may be isolated from natural sources or prepared synthetically, for example, by incubation of isolated soluble assemblies of amyloid A, such as, for example Aβ*56, and one or NMDA receptors or one or more NMDA receptor subunits. Such complexes are useful, for example, as positive controls in any of a variety of assays, including, but not limited to, methods to detect to coprecipitation of an amyloid β and a NMDA receptor and methods to detect agents that alter the antagonistic effect soluble assemblies of amyloid β on NMDA receptor function. The present invention also includes compositions of isolated complexes of soluble assemblies of amyloid β and NMDA receptors.

Isolated complexes of soluble assemblies of amyloid β protein and the NMDA receptor may serve as an antigen or vaccine to immunize an animal to elicit an immune response. The preparation and use of such antigens and vaccines is well known in the art. Immunization may be accomplished in the presence or absence of an adjuvant, e.g., Freund's adjuvant. Booster immunizations may be given at intervals, for example, at two to eight weeks.

As used herein, “isolated” refers to material removed from its original environment (e.g., the natural environment if it is naturally occurring), and thus is altered “by the hand of man” from its natural state.

Any of the methods described in WO 2004/031400 (“Amyloid Beta-Derived Diffusible Ligands (ADDLS), ADDL-Surrogates, ADDL-Binding Molecules, and Uses Thereof”) and Lacor et al., Neurobiology of Disease 23(45):101991-10200, 2004 (“Synaptic Targeting by Alzheimer's-Related Amyloid β Oligomers”) may be used in the present invention.

The present invention is illustrated by the following examples. It is to be understood that the particular examples, materials, amounts, and procedures are to be interpreted broadly in accordance with the scope and spirit of the invention as set forth herein. All headings are for the convenience of the reader and should not be used to limit the meaning of the text that follows the heading, unless so specified.

### EXAMPLES

### Example 1

### Aβ*56 is an NMDA Receptor Antagonist

Amyloid-β (Aβ) proteins accumulate in Alzheimer's disease (AD), but the mechanism by which they disrupt cognitive function is unclear. This example shows that Aβ*56 (Aβ star 56), a 56-4β complex of An molecules, specifically binds NR1 and NR2A subunits of NMDA receptors in Tg2576 and AD brains. Purified Aβ*56 increases NMDA-mediated spontaneous circuit activity and inhibits NMDA evoked currents in cultured neurons. From these result, it can be concluded that Aβ*56 is an NMDA receptor antagonist. Because NMDA receptors are critical mediators of long lasting synaptic plasticity and memory, the data define a new mechanism by which Aβ may impair cognitive function associated with AD.

The effects of synthetic soluble Aβ oligomers and Aβ oligomers secreted by cultured cells include impairment of neuronal survival (Lambert et al., Proc Natl Acad Sci USA 95, 6448 (1998); Kayed et al., Science 300, 486 (2003)), inhibition of long-lasting synaptic plasticity (Wang et al., Brain Res 924, 133 (2002); Walsh et al., Nature 416, 535 (2002); and Wang et al., J Neurosci 24, 3370 (2004)), and disruption of behaviour (Cleary et al., Nat Neurosci 8, 79 (2005)), as well as up-regulation of the synaptic immediate-early gene Arc (Lacor et al., J Neurosci 24, 10191 (2004)), and endocytosis of NMDA receptors (Snyder et al., Nature Neuroscience 8, 1051 (2005)). However, the effects of endogenous soluble Aβ protein assemblies generated in vivo have only recently been elucidated (WO 2006/047254). The endogenous Aβ assembly, Aβ*56, correlates strongly with spatial memory in Tg2576 mice and disrupts cognitive function in rats, in the absence of neuronal loss or amyloid plaque deposition (WO 2006/047254). Because glutamate receptors are important elements in synaptic plasticity and memory (Collingridge, Nature 330, 604 (1987); Malinow et al., Annu Rev Neurosci 25, 103 (2002); and Dudai, “Memory from A to Z: Keywords, Concepts and Beyond” (Oxford University Press, Oxford, 2002)), and down-regulation of Arc and other synaptic genes critical for memory consolidation occurs in Tg2576 and other APP transgenic mice (Dickey et al., J Neurosci 23, 5219 (2003); Palop et al., Proc Natl Acad Sci USA 100, 9572 (2003)), with this example the possibility that Aβ*56 impairs memory by interacting directly with glutamate receptors was examined.

### Materials and Methods

Transgenic animals. Tg2576 mice (Hsiao et al., Science 274, 99-102 (1996)) were the offspring of mice backcrossed successively to B6SJLF1 breeders.

Human brain tissue. Frozen specimens of cerebral cortex were obtained from three Alzheimer's disease patients and two cognitively intact control subjects from the Rush Alzheimer's Disease Center (Chicago, Ill.), and one Alzheimer's disease patient from the Regions Hospital Alzheimer's Treatment and Research Center (St. Paul, Minn.).

Cultured neurons. Cultures of cortical neurons from P1 Sprague Dawley rat pups were prepared according to previously published protocols (Dubinsky, J Neurosci 13, 623-631 (1993)). Dissections of cortical mantels excluded meninges, hippocampus, septal regions, and basal ganglia. Cortical neurons were plated at an approximate density of 50,000-100,000/centimeters squared (cm²) onto preplated cortical astroglial feeder layers (Dubinsky, J Neurosci 13, 623-631 (1993)) on Themanox plastic coverslips (Electron Microscopy Sciences, Ft. Washington, Pa.) in Minimal Essential Medium without glutamine, supplemented with 10% NuSerum (Life Technologies, Grand Island, N.Y.), 27 millimolar (mM) glucose, 50 units/milliliter (U/ml) penicillin, and 50 micrograms/milliliter (μg/ml) streptomycin and maintained at 37° C. in 5% CO₂. Cultures were used between twelve and sixteen days in vitro at a time when synaptic networks were well developed (Dichter, Brain Res 149, 279-293 (1978)).

Antibodies. The following primary antibodies were used: 6E10 and 4G8 [1:100-10,000] respectively against Aβ1-17 and Aβ17-25 (Signet Laboratories, USA), APPCter-C17 [1:5000] against APP C-terminus (Sergeant et al., J. Neurochem 81(4):663-72 (2002)), antibodies raised against PSD95, NR1, NR2 subunits (A-D), GluRs [1:200] (SantaCruz Biotechnologies Inc, USA).

Protein extractions. Soluble, extracellular-enriched fractions were generated from hemi-forebrains harvested in 500 microliter (μl) of solution containing 50 millimolar (mM) Tris-HCl (pH 7.6), 0.01 % NP-40, 150 mM NaCl, 2 mM EDTA, 0.1 % SDS, 1 mM phenylmethylsulfonyl fluoride (PMSF), and protease inhibitor cocktail (Sigma). Soluble, extracellular enriched proteins were collected from mechanically homogenized lysates (1 milliliter (ml) syringe, gauge 20 needle [ten repeats]) following centrifugation for five minutes at 3,000 rpm. Membrane-enriched fractions were generated from hemi-forebrains harvested in 500 μl of solution containing 50 mM Tris-HCl (pH 7.6), 0.1% NP-40, 150 mM NaCl, 2 mM EDTA, 1% SDS, 1 mM PMSF, 2 mM 1,10-PTH and protease inhibitor cocktail (Sigma). Lysates were mechanically homogenized (1 ml syringe and needle, guage 20 [ten repeats]) and centrifuged for 90 minutes at 13,000 rpm. Membrane-associated proteins were generated from the pellets re-suspended with 500 μl of buffer (50 mM Tris-HCl[pH 7.4], 150 mM NaCl, 0.5% Triton X-100, 1 mM EGTA, 3% SDS, 1% deoxycholate, 1 mM of PMSF) following centrifugation for 90 minutes at 13,000 rpm.

All supernatants were clarified by centrifuging for 90 minutes at 13,000 rpm prior to western blot analysis. Protein amounts were determined (BCA Protein Assay, Pierce). Western blot and immunoprecipitations were performed as described in WO 2006/047254.

Immunoaffinity chromatography. Tg2576 brain proteins remaining in the supernatant following tissue lysis with RIPA buffer and centrifugation for 90 minutes at 13,000 rpm at 4° C. were incubated overnight with affinity columns packed with 200 micrograms (μg) of purified antibody (6E10 or 4G8). Columns were created using the Seize™ Primary Immunoprecipitation Kit (Pierce) following the manufacturer's instructions.

Size-exclusion chromatography (SEC). Immunoaffinity purified protein extracts were loaded on Tricom Superdex® 75 columns (Amersham Life Sciences, Piscataway, N.J.) and run at a flow rate of approximately 0.3 milliliter per minute (ml/min). Fractions of 500 μl of eluate in 50 mM Ammonium Acetate, pH 8.5, were diluted 1:100 or 1:750 in neurophysiology experiments, or were concentrated using a vacuum system (VacuFuge™, Brinkmann-Eppendorf) and analyzed by silver staining.

Silver staining. Following SEC fractionation and SDS polyacrylamide gel electrophoresis (SDS-PAGE), gels were subjected to silver staining to control the purity of the samples. For sensitivity purposes, proteins were stained using the SilverXpress® Silver Staining Kit (Invitrogen™ Life Technologies, USA) with adapted protocol. Changes are as follows: all washing steps were repeated four times and gels were incubated in Developing Solution for fifteen minutes.

Calcium imaging. Neuronal cultures were incubated in the Ca²⁺-indicator dye Fluo-4 AM (31 μg/ml) and pluronic acid (2.6 mg/ml) for 30 minutes at room temperature. Calcium indicator dye fluorescence was monitored with 488 nanometer (nm) excitation, a 500 nm long-pass barrier filter and confocal microscopy (Odyssey scanner, Noran, Middleton, Wis.). Cultures were perfused with HEPES-buffered saline equilibrated with air, pH 7.4. Glycine was not added to the perfusate. To quantify neuronal Ca²⁺ responses, Ca²⁺ fluorescence was measured from single neurons or clusters of neurons. Baseline fluorescence was normalized to one and the area between the curve and the baseline calculated using Origin software analysis routines (OriginLab Corporation, Northampton, Mass.).

Whole cell patch recordings. Neuronal cultures were perfused with HEPES-buffered saline supplemented with 10 μM glycine and 200 nanomolar (nM) tetrodotoxin. Individual neurons were stimulated by focal ejection of 50 μM NMDA and 10 μM glycine. Cells were whole cell voltage clamped and NMDA-evoked inward currents recorded at a holding potential of −35 mV. The HEPES-buffered saline solution contained 135.5 mM NaCl, 3.0 mM KCl; 2.0 mM CaCl₂; 1.0 mM MgSO₄; 0.5 mM NaH₂PO₄; 15.0 mM D-glucose; 10 mM HEPES; pH 7.4. The intracellular pipette solution contained 5.0 mM Na-methanesulfonate; 128.0 mM K-methanesulfonate; 2.0 mM MgCl₂; 5.0 mM K-EGTA; 1.0 mM glutathione; 2.0 mM MgATP; 0.2 mM NaGTP; 5.0 mM HEPES; pH 7.4.

### Results and Discussion

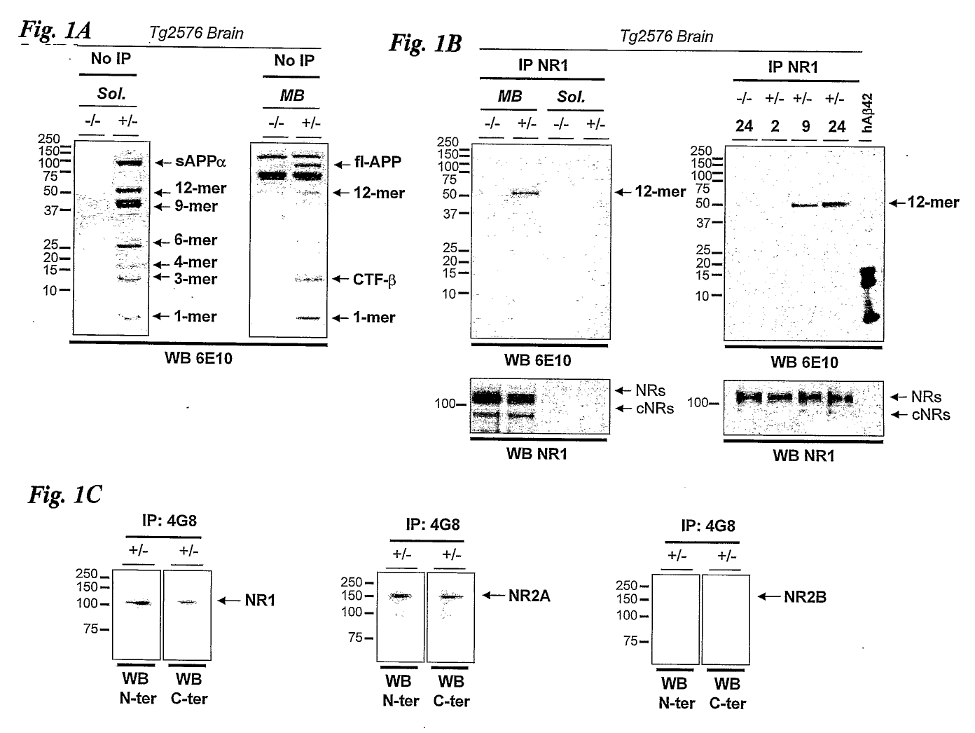

In Tg2576 mice, Aβ*56 is found in soluble, extracellular-enriched protein fractions in the brain (WO 2006/047254). To test the hypothesis that Aβ*56 is a receptor ligand, membrane-enriched fractions were examined by western blotting with 6E10 monoclonal antibodies (raised against Aβ1-16). An immunoreactive band corresponding in molecular weight to Aβ*56 was found, in addition to the expected bands corresponding to full length APP and its derivatives CTF-β and monomeric Aβ (FIG. 1A). Importantly, although Aβ*56 is one of several species of Aβ oligomers in the soluble, extracellular enriched protein fraction, Aβ*56 was the only Aβ species detected after modest film exposure times, indicating it is the dominant Aβ species associated with cell membranes. The possibility that the 56 kilodalton (kD) band was a degradation product of APP was excluded by the demonstration that 22C11 (raised against the N-terminal region of APP) and APP17Cter (raised against the C-terminal regions of APP) failed to bind the 56-kD species.

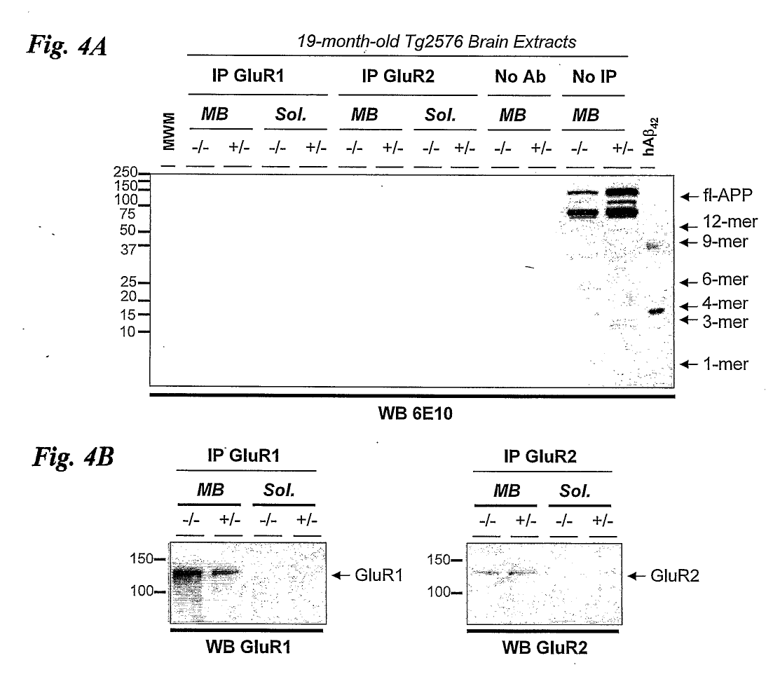

Next, cross-immunoprecipitations were performed against ionotropic glutamate receptor subunits and AB in membrane-enriched Tg2576 brain extracts. When proteins were captured with antibodies directed against the NMDA receptor subunit NR1, Aβ*56, but no other Aβ species, was detected with either 6E10 (FIG. 1B) or 4G8 antibodies. Aβ*56 was not immunoprecipitated by NR1 antibodies in soluble, extracellular-enriched extracts containing similar amounts of Aβ*56 (FIGS. 1A and 1B). This is because NR1 immunoreactivity after NR1 immunoprecipitation was present only in the membrane-enriched extracts, as expected (FIG. 1B). Aβ*56 was immunoprecipitated with NR1 antibodies in nine and twenty-four month, but not two month Tg2576 brain extracts (FIG. 1B), which is consistent with the absence of Aβ*56 in less than six-month-old mice exhibiting normal spatial memory (WO 2006/047254). NR2A antibodies also immunoprecipitated Aβ*56, and were considerably more effective than NR2B antibodies in immunoprecipitating Aβ*56. The 4G8 antibody (raised against Aβ17-24) immunoprecipitated NR1, NR2A, but not NR2B, receptor subunits from membrane-enriched Tg2576 brain extracts (FIG. 1C), confirming immunoprecipitation experiments with NR1, NRA and NR2B antibodies. When proteins were captured with antibodies to the AMPA receptor subunits, GluR1 and GluR2, neither Aβ*56 nor any other AB oligomers were immunoprecipitated (FIGS. 4A-4B), indicating that Aβ*56 does not bind AMPA receptors.

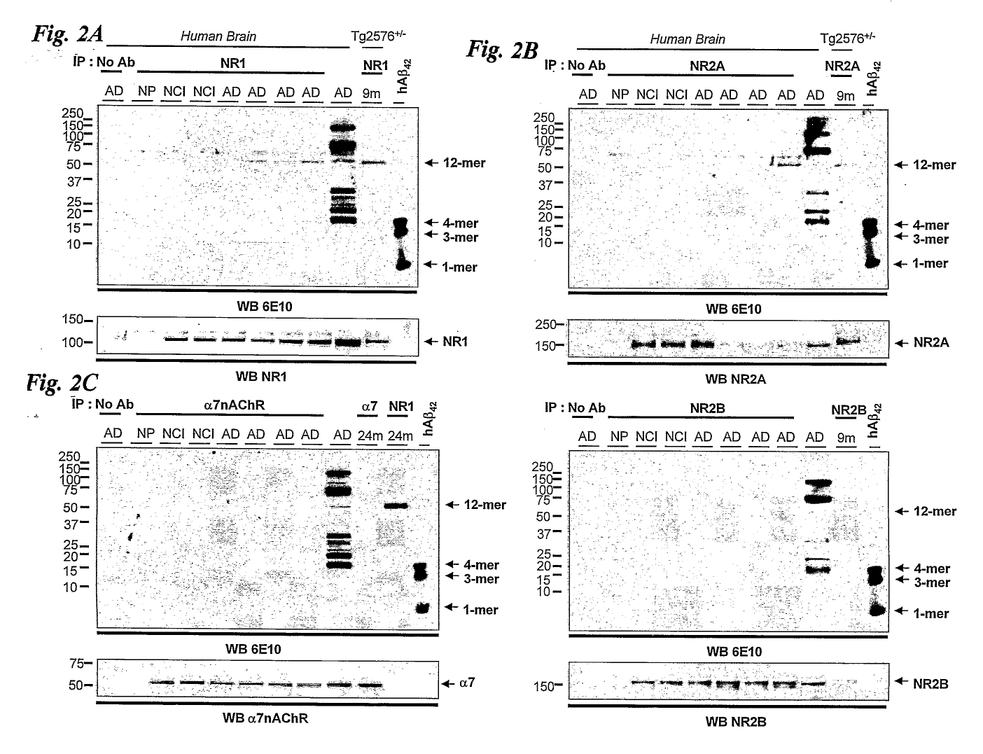

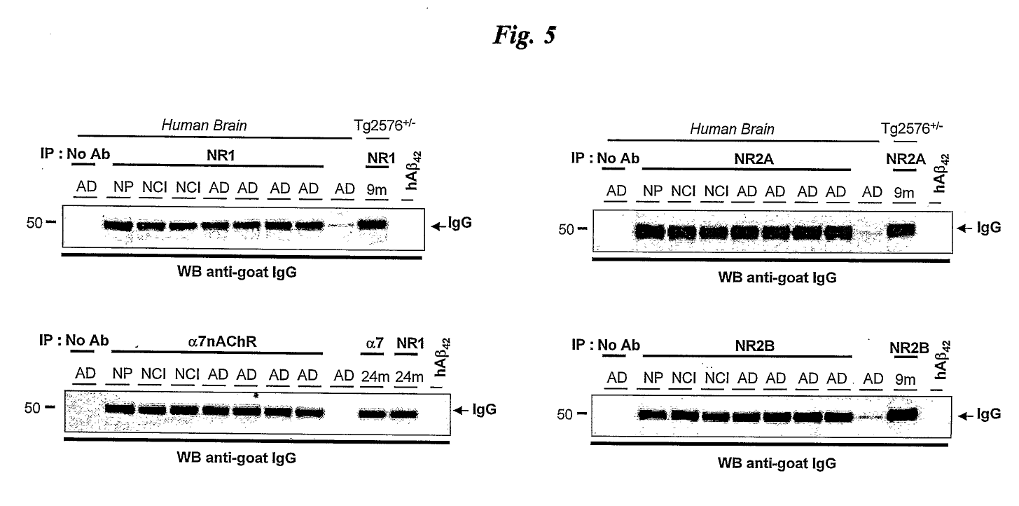

The interaction of Aβ assemblies and ionotropic glutamate receptors in brain tissue from patients with AD and control individuals without dementia was examined. NR1, NR2A and, to a significantly lesser extent, NR2B antibodies immunoprecipitated a 56-kD 6E10-immunoreactive protein co-migrating with Aβ*56 in brain tissue samples from all four patients with Alzheimer's disease, but in neither of two samples from control individuals with no cognitive impairment (NCI) (FIGS. 2A and 2B). The unequal levels of NR2A subunits were not due to inconsistencies in loading samples (FIG. 5), and therefore reflected actual receptor subunit levels in the brain specimens. These data indicate that Aβ*56 or an Aβ*56-like molecule binds NMDA receptors selectively in Alzheimer's disease.

Soluble Aβ oligomers have been postulated to bind α7-nicotinic acetylcholine (nACh) receptors (Dineley et al., J Neurosci 21, 4125 (2001)) and thereby mediate increased endocytosis of NMDA receptors (Snyder et al., Nature Neuroscience 8, 1051 (2005)). Whether Aβ*56 binds α7-nACh receptors was therefore examined. Aβ*56 was not immunoprecipitated from membrane-enriched Tg2576 brain extracts using anti-α7-nACh receptor antibodies (FIG. 2C). The receptors were detected with α7-nACh receptor antibodies in the immunoprecipitated material, excluding failure to immunoprecipitate the receptors as a possible explanation for the absence of Aβ*56. The data indicate that Aβ*56 does not bind α7-nACh receptors in human or Tg2576 brain tissue.

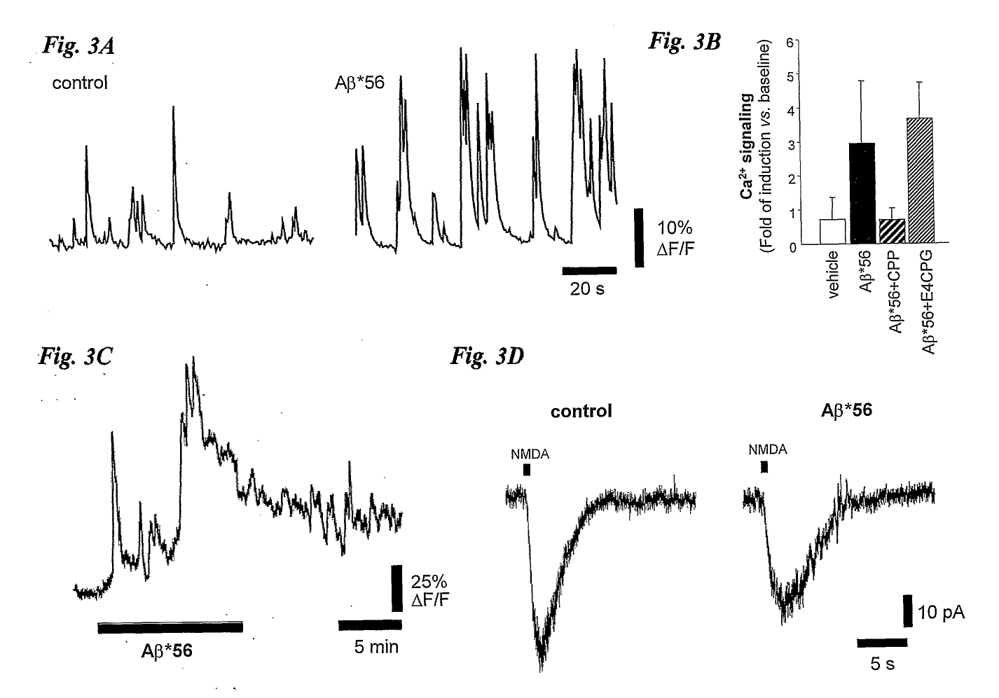

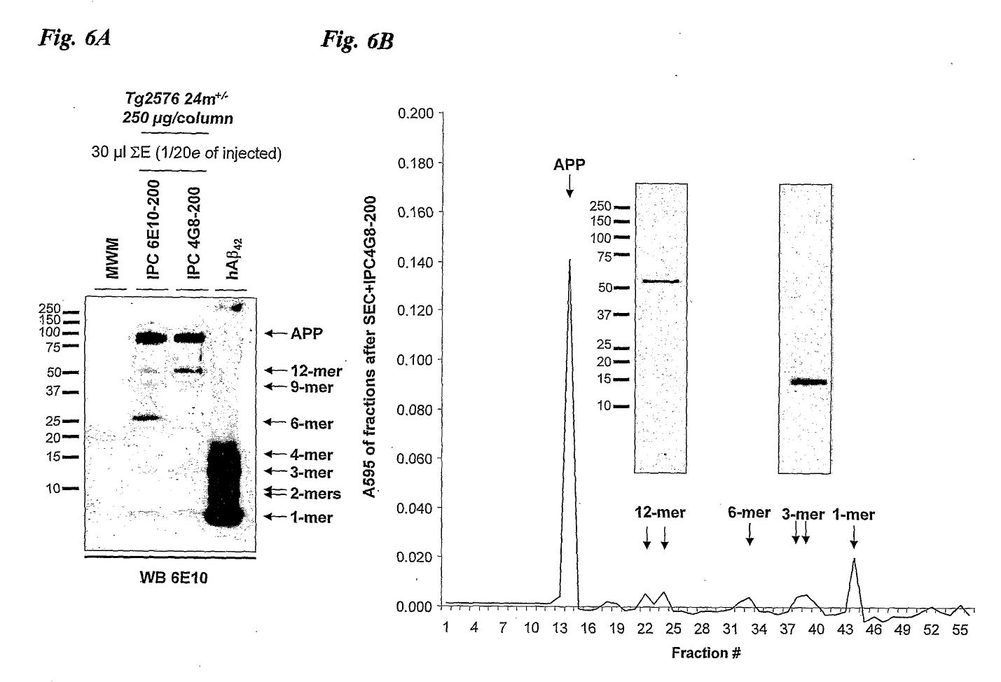

To ascertain whether the physical interaction of Aβ*56 and NMDA receptors is functionally relevant, purified Aβ*56 from Tg2576 brain was applied to primary cultured neurons and measured changes in intraneuronal ionic calcium (Ca²⁺) and NMDA evoked currents. Aβ*56 was purified using immunoaffinity chromatography followed by size-exclusion chromatography, which yielded preparations of Aβ*56 that ran as a single band on silver-stained gels (FIGS. 6A and 6B). Changes in intracellular Ca²⁺ were monitored in primary cultured neurons in response to external applications of Aβ*56, and quantified Ca²⁺ concentrations by integrating Ca²⁺ indicator dye fluorescence over a 100 second period using confocal microscopy. The addition of 7-8.5 nM Aβ*56 led to an approximately 3-fold (2.95±1.83, n=26 cell clusters from five cultures) elevation of the resting Ca²⁺ signal (FIGS. 3A and 3B), which was completely abolished when the competitive NMDA receptor antagonist, (+/−)-3-(2-carboxypiperazin-4-yl)-propyl-1-phosphonic acid (CPP), was co-applied with Aβ*56 (FIG. 3B). In contrast, the Group I/Group II mGluR antagonist (RS)-α-Ethyl-4-carboxyphenylglycine (E4CPG) failed to block the increase in Ca²+triggered by Aβ*56 (FIG. 3B). In single cell measurements, Aβ*56 induced a rapid Ca²⁺ increase within seconds that remained well above baseline for longer than twenty-five minutes (min) (FIG. 3C). The increase in Ca²⁺ remained elevated for at least ten minutes after Aβ*56 was washed out of the culture dish. These results indicate that Aβ*56 acts at the NMDA receptor within seconds to increase the circuit activity of neuronal networks in culture, and suggest that the Aβ*56-NMDA receptor complex remains functionally active at the neuronal membrane for relatively long periods of time after initial exposure (that is, at least twenty-five minutes). The increased electrical activity of the neuronal network may be explained in a couple of ways: Aβ*56 may be an NMDA receptor agonist which depolarizes neurons expressing NMDA receptors, in turn leading to synaptic activation of connected neurons; or Aβ*56 may be an NMDA receptor antagonist in a network in which inhibitory interneurons are activated by neurons expressing NMDA receptors, inhibition of which leads to an overall increase in network activity.

To test the effects of Aβ*56 on NMDA-evoked currents directly, whole cell patch clamp recordings were preformed on cultured neurons (FIG. 3D). The addition of 1 nM Aβ*56 significantly reduced NMDA-evoked currents to 70±8% of control values (n=15, p<0.005) within five minutes of exposure to Aβ*56, while application of vehicle alone did not alter NMDA-evoked currents (106±7% of control, n=7). These results indicate that Aβ*56 is an NMDA receptor antagonist at the concentration (1 nM) tested, which is the concentration of Aβ measured by standard enzyme-linked immunosorbent assays in human cerebral spinal fluid (Walsh et al., Nature 416, 535 (2002)). There are several possible mechanisms by which Aβ*56 could inhibit NMDA receptor function. It could be a competitive NMDA receptor antagonist. It could induce NMDA receptor endocytosis, probably not via the α7-nACh receptor (Snyder et al., Nature Neuroscience 8, 1051 (2005)), but possibly by mimicking the priming of NMDA receptor endocytosis by glycine (Nong et al., Nature 422, 302 (2003)). Or, it could act by a combination of these activities. It will be important in future studies to elucidate the mechanism by which Aβ*56 antagonizes NMDA receptor function.

Aβ*56 is the sole endogenous Aβ protein complex whose disruptive effects on cognitive function have been described (WO 2006/047254). The binding of Aβ*56 to synaptic NMDA receptors in Tg2576 and AD brain and its identification as an NMDA receptor antagonist at physiologic, nanomolar concentrations suggest that it functions like a pharmacological agent in the brain. It is proposed that Aβ*56 disrupts memory and cognitive function by altering the intrinsic physiological function of NMDA receptors, which are critical mediators of long-lasting synaptic plasticity (Collingridge, Nature 330, 604 (1987); Collingridge et al., Nat Rev Neurosci 5, 952 (2004); and Bliss and Collingridge, Nature 361, 31 (1993)) and memory (Dudai, “Memory from A to Z: Keywords, Concepts and Beyond” (Oxford University Press, Oxford, 2002); Morris et al., Nature 319, 774 (1986)). The reversibility of pre-existing memory deficits in Tg2576 and other APP transgenic mice (Kotilinek et al., J Neurosci 22, 6331 (2002); Dodart et al., Nat Neurosci 5, 452 (2002)), and the transience of cognitive disruption induced by Aβ*56 in normal rats (Cleary et al., Nat Neurosci 8, 79 (2005)), is consistent with the pharmacological properties of Aβ*56. The selective down-regulation of genes important for long-lasting synaptic plasticity in Tg2576 and other APP transgenic mice (Dickey et al., J Neurosci 23, 5219 (2003); Palop et al., Proc Natl Acad Sci USA 100, 9572 (2003)) may be a result of the NMDA receptor antagonistic properties of Aβ*56. The present example defines a mechanism by which AS causes reversible neuronal dysfunction rather than irreversible structural degeneration. Targeting reversible neuronal dysfunction with drugs aimed at the interaction between Aβ*56 and NMDA receptors may prevent dementia in persons at risk for AD and restore brain function in the early stages of Alzheimer's disease.

### Example 2

### Antibodies to Aβ block Aβ*-Mediated Inhibition of NMDA-Evoked Currents

Candidate anti-Aβ clones will be screened comprehensively using methods that ensure that the anti-Aβ* monoclonals specifically recognize natively folded Aβ*56, and do not bind fibrillar or monomeric Aβ. Dot blot methods followed by confirmatory liquid-phase immunoprecipitation and immunoblotting experiments can be used for this purpose. Direct liquid phase ELISA methods can also be used. The dot blot method is advantageous due to its rapid throughput and minimal potential for steric hindrance preventing detection of suitable clones. The ELISA method is useful due to its ability to detect natively folded Aβ*56 directly.

Various methods may be used to screen candidate monoclonals for antibodies that specifically detect Aβ*56. In the Dot Blot assay, Aβ*56, synthetic monomeric Aβ(1-42), soluble Aβ(1-42) oligomers and fibrillar Aβ(1-42) can be spotted at known concentrations on nitrocellulose or nylon filters. The filters can be overlaid with candidate monoclonals. Clones that selectively stain Aβ*56 at low concentrations can be selected. In the Western Blot assay, which is a confirmatory test for the Dot Blot assay, Aβ*56, synthetic monomeric Aβ(1-42), soluble Aβ(1-42) oligomers and fibrillar Aβ(1-42) can be size-fractionated by polyacrylamide gel electrophoresis and transferred to nitrocellulose or nylon filters. The filters can be overlaid with candidate monoclonals. Clones that selectively stain Aβ*56 but no other forms of Aβ can be selected. In the liquid phase ELISA method, monoclonal anti-Aβ antibodies 6E10 or 4G8 can be immobilized onto the wells of plastic plates, overlaid with Aβ*56. Candidate monoclonals can be applied to wells. Clones that bind Aβ*56 can be detected with goat anti-mouse antibodies conjugated to a fluorescent marker.

To generate anti-Aβ*monoclonals, mice will be immunized with purified Aβ*56 from the brains of Tg2576 mice greater than six months old, AD patients, or Down syndrome patients, or with synthetic AB oligomers that include species which are 56 kDa. Aβ*56 can be purified by immunoaffinity chromatography followed by size-exclusion chromatography so that it runs as a single band on silver stained gels. Biochemical methods can also be used to purify Aβ*56, taking advantage of the stability of Aβ*56 in 8M urea, which denatures most globular proteins.

To determine that the purified immunogen is biologically active, it can be assayed for its ability to inhibit NMDA-evoked currents in cultured neurons, prior to injection as an immunogen. However, this is not an essential step. It is expected that these methods will successfully lead to the generation of specific antibodies to Aβ*56, particularly since multimerized proteins tend to be better immunogens than monomeric proteins, because they crosslink immunoglobulins on B-cells.

It is possible that Aβ*56 in human brain (from AD patients) and mouse brain (from Tg2576 mice) may differ subtly in conformation. Therefore, both natively folded Aβ*56 purified from human AD brains and Tg2576 mouse brain tissue can be used to screen monoclonals. The results will generate a two by two catalogue of clones showing specific recognition of AD-Aβ*56, Tg2576-Aβ*56, both AD-Aβ*56 and Tg2576-Aβ*56, or neither protein complex. This catalogue will aid in selecting the most appropriate anti-Aβ* monoclonals for use in humans.

Assessing the ability of Aβ*-monoclonals to block Aβ*-mediated inhibition of NMDA-evoked currents in cultured rat cortical neurons and human neuronal cell lines. Confirmation of the ability of anti-Aβ* monoclonals to block the inhibitory effects of Tg2576-Aβ*56 or AD-Aβ*56 on NMDA-evoked currents in cultured cortical neurons may be accomplished using the methods described in Example 1, assessing the effects of Tg2576-Aβ*56 on NMDA-evoked currents. These experiments will ensure that the anti-Aβ* monoclonals specifically block the interaction of Tg2576-Aβ*56 or AD-Aβ*56 with rat and human NMDA receptors. Anti-Aβ* monoclonals which have been functionally and biochemically validated may then undergo further screening at lower concentrations to define dose-effect curves, which will serve to identify the most potent monoclonal inhibitors of the effects of Aβ*56 on memory, cognitive function and NMDA receptor function.

### Example 3

### NMDA Receptor Function Determined by Ca²⁺ Fluorescence Imaging

The assay will use Ca²⁺ fluorescence imaging to monitor the electrical activity of neurons in culture. The assay determines the effective dose of Aβ*56 in binding to NMDA receptors. An advantage of the assay is that the effect of Aβ*56 on NMDA receptor function can be determined quickly and easily.

Cultured neurons. Cultures of cortical neurons from rat pups are prepared according to established protocols. Neurons are co-cultured with glial cells on glass coverslips. Cultured neurons between twelve and sixteen days in vitro are used, when synaptic networks are well developed and spontaneous electrical activity occurs.

Calcium imaging. Cultured neurons on coverslips are incubated in the Ca²⁺-indicator dye Fluo-4 AM (31 μg/ml) and pluronic acid (2.6 mg/ml) for 30 minutes at room temperature. The coverslips are then rinsed and placed in a Petri dish containing HEPES-buffered saline equilibrated with air, pH 7.4. Calcium indicator dye fluorescence is monitored with 488 nm excitation, a 500 nm long-pass barrier filter and either confocal microscopy or epifluorescence microscopy.

Analysis. Calcium fluorescence is measured from single neurons or clusters of neurons to quantify neuronal activity. Baseline fluorescence is normalized and the area between the curve and the baseline calculated using Origin software analysis routines. The resulting integrated Ca²⁺ signal is used as a measure of neuronal activity.

Experimental protocol for determining the Aβ*56 dose-response relation. Baseline neuronal activity in control solution will be determined using the Ca²⁺ fluorescence assay. The concentration of Aβ*56 in the solution will then be raised stepwise and neuronal activity will be determined at each Aβ*56 concentration. A vehicle control will also conducted to ensure that changes in neuronal activity are not due to the buffer solution that contains the Aβ*56.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

For any method disclosed herein that includes discrete steps, the steps may be conducted in any feasible order. And, as appropriate, any combination of two or more steps may be conducted simultaneously.

All headings are for the convenience of the reader and should not be used to limit the meaning of the text that follows the heading, unless so specified.

1. A method of detecting a neurodegenerative disease and/or cognitive disorder in a subject, the method comprising:
obtaining a sample from the subject;
immunoprecipitating the sample with an antibody to a NMDA receptor;
wherein the coprecipitation of amyloid β along with the NMDA receptor indicates the subject has a neurodegenerative disease and/or cognitive disorder.

2. The method of claim 1 wherein the neurodegenerative disease and/or cognitive disorder is a presymptomatic neurodegenerative disease and/or cognitive disorder and wherein the coprecipitation of amyloid β along with the NMDA receptor indicates the subject has a presymptomatic neurodegenerative disease and/or cognitive disorder disease.

3. The method of claim 1, wherein the antibody to an NMDA receptor is an antibody that binds to a NMDA receptor subunit selected from the group consisting of NR1, NR2A, and NR2B.

4. A method of inhibiting NMDA receptor function, the method comprising contacting a NMDA receptor with a soluble assembly of amyloid β protein.

5. A method of screening for an agent that alters the antagonistic effect of a soluble assembly of amyloid β protein on NMDA receptor function, the method comprising:
contacting a NMDA receptor with the agent and a soluble assembly of amyloid β protein;
determining NMDA receptor function for the NMDA receptor contacted with the agent and the soluble assembly of amyloid β protein;
comparing NMDA receptor function for the NMDA receptor contacted with the agent and the soluble assembly of amyloid β protein to NMDA receptor function for a NMDA receptor contacted with the soluble assembly of amyloid β protein and not contacted with the agent;
wherein a difference in the level of NMDA receptor function in the NMDA receptor contacted with the agent and the soluble assembly of amyloid β protein compared to NMDA receptor function in the NMDA receptor contacted with the soluble assembly of amyloid β protein and not contacted with the agent indicates the agent alters the antagonistic effect of the soluble assembly of amyloid β protein on NMDA receptor function.

6. A method of screening for an agent for the treatment of neurodegenerative disease and/or cognitive disorder, the method comprising:
contacting a NMDA receptor with the agent and a soluble assembly of amyloid β protein;
determining NMDA receptor function for the NMDA receptor contacted with the agent and the soluble assembly of amyloid β protein;
comparing NMDA receptor function for the NMDA receptor contacted with the agent and the soluble assembly of amyloid β protein to NMDA receptor function for an NMDA receptor contacted with the soluble assembly of amyloid β protein and not contacted with the agent;
wherein an altered level of NMDA receptor function in the NMDA receptor contacted with the agent and the soluble assembly of amyloid β protein compared to the NMDA receptor function of the NMDA receptor contacted with the soluble assembly of amyloid β protein and not contacted with the agent indicates the agent as an agent for the treatment of neurodegenerative disease and/or cognitive disorder.

7. The method of claim 5, wherein the agent inhibits the antagonistic effect of the soluble assembly of amyloid β protein on NMDA receptor function.

8. The method of claim 5, wherein NMDA receptor function is determined by whole cell patch clamp recording.

9. The method of claim 5, wherein NMDA receptor function is determined by Ca⁺² fluorescence imaging.

10. A method of treating a neurodegenerative disease and/or a cognitive disorder in a subject, the method comprising administering to the subject an effective amount of an agent that alters the antagonistic effect of a soluble assembly of amyloid β protein on the NMDA receptor.

11. The method of claim 1, wherein the neurodegenerative disease and/or cognitive disorder is Alzheimer's disease.

12. The method of claim 1, wherein the soluble assembly of amyloid β protein has a molecular weight of about 56 kDa as measured by SDS polyacrylamide gel electrophoresis.

13. The method of claim 1, wherein the soluble assembly of amyloid β protein comprises a dodecamer of amyloid β proteins.

14. The method of claim 1, wherein the soluble assembly of amyloid β protein is Aβ*56.

15. An isolated Aβ*56/NMDA receptor complex.

16. An agent that alters the inhibitory effect of a soluble assembly of amyloid β protein on the NMDA receptor.

17. An agent identified by claim 5.

18. An antibody that binds to a soluble assembly of amyloid β protein and prevents the formation of an amyloid β/NMDA receptor complex.

19. An antibody that binds to a NMDA receptor and prevents the formation of an amyloid β/NMDA receptor complex.

20. A method of treating a neurodegenerative disease and/or a cognitive disorder in a subject, the method comprising administering to the subject an effective amount of an antibody of claim 18.
